(19) United States
(12) Patent Application Publication
(10) Pub. No.: US 2015/0224190 A1
(43) Pub. Date: Aug. 13, 2015

(54) Combination of a phosphoinositide 3-kinase inhibitor and an inhibitor of the IL-8/CXCR interaction

(71) Applicants: Mohamed Bentires-Alj, (US); Adrian Britschgi, (US); Thomas Radimerski, (US); Rita Andraos-Rey, (US)

(72) Inventors: Mohamed Bentires-Alj, Kembs-Loechle (FR); Adrian Britschgi, Zurich (CH); Thomas Radimerski, Tecknau (CH); Rita Andraos-Rey, St. Louis (FR)

(21) Appl. No.: 14/410,598

(22) PCT Filed: Jul. 3, 2013
(86) PCT No.: PCT/EP2013/064083

§ 371 Date: Dec. 23, 2014

(30) Foreign Application Priority Data
Jul. 6, 2012 (EP) 12175434.5

(51) Int. Cl. A61K 39/395; A61K 31/436; A61K 31/5377

(52) U.S. Cl. CPC A61K 39/3955; A61K 31/5377; A61K 31/436; A61K 2039/505

(57) ABSTRACT

The invention relates to a pharmaceutical combination which comprises (a) a phosphoinositide 3-kinase inhibitor compound and (b) a compound which inhibits the interaction between IL-8 and at least one of its receptors for the treatment of a proliferative disease, especially a solid tumor disease; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

The invention relates to a pharmaceutical combination which comprises (a) a phosphoinositide 3-kinase (PI3K)/mammalian target of rapamycin (mTOR) inhibitor compound and (b) a compound or neutralizing antibody which inhibits interleukin-8 (IL-8) and/or the interaction between interleukin-8 (IL-8) and its receptor, the Chemokine (C-X-C motif) receptor (CXCR), or small molecule inhibitor or antibody antagonist of said receptor, and optionally at least one pharmaceutically acceptable carrier for simultaneous, separate or sequential use, in particular for the treatment of a proliferative disease, especially a proliferative disease in which the PI3K/Akt pathway is dysregulated; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

The rapid development of highly specific inhibitors targeting key signaling pathways (e.g., PI3K/mTOR) has created much excitement in the cancer research community. The clinical efficacy and low toxicity of some of these rationally designed therapies raised the hope for a new era for the treatment of cancer. Unfortunately, single-agent targeted cancer therapy is often thwarted by adaptive resistance, tumor recurrence and an ineluctable downhill course. A better understanding of the crosstalks between oncogenic signaling pathways is fundamental to curb resistance to targeted therapy and should lead to novel, hopefully curative, combination therapies.

The phosphatidylinositol 3-kinase (PI3K) pathway, a central regulator of diverse normal cellular functions, is often subverted during neoplastic transformation. Mechanisms of activation of the PI3K pathway in cancer include: mutation and/or amplification of PIK3CA, the gene encoding p110α, the alpha catalytic subunit of the kinase; loss of expression of PTEN, the phosphatase that reverses PI3K activity; activation downstream of oncogenic receptor tyrosine kinases; and Akt amplification. By decreasing cell death, increasing cell proliferation, migration, invasion, metabolism, angiogenesis and resistance to chemotherapy, an aberrant PI3K pathway provides cancer cells with a competitive advantage. Not surprisingly, the PI3K/Akt/mTOR cascade is an attractive therapeutic target and several inhibitors of this pathway are currently in clinical trials.

Using several cell lines and primary tumor models of triple-negative breast cancer, the present inventors found that PI3K/mTOR inhibition elicited a vicious positive feedback loop by activating JAK2-STAT5 signaling which eventually induced secretion of IL-8. After a series of extensive further experiments, the inventors now found that a direct inhibition of the interaction of IL-8 and its receptors, in combination with the inhibition of the PI3K/Akt/mTOR pathway, reduces tumor seeding and metastasis.

Building on insights gained from mechanistic understanding of PI3K/mTOR inhibition, the present inventors demonstrated the therapeutic efficacy of combined inhibition of the PI3K/mTOR and of the interaction of IL-8 and its receptors. Indeed combined inhibition of PI3K/mTOR of the interaction of IL-8 and its receptors reduced tumor growth and seeding as well as metastasis.

WO2006/122806 describes imidazoquinoline derivatives, which have been described to inhibit the activity of lipid kinases, such as PI3-kinases. Specific imidazoquinoline derivatives which are suitable for the present invention, their preparation and suitable pharmaceutical formulations containing the same are described in WO2006/122806 and include compounds of formula I

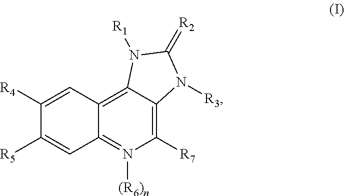

wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl;
pyrazolyl; and triazolyl;

### R₂ is O or S;

R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen;
quinoxalinyl; or phenyl substituted with alkoxy
R₅ is hydrogen or halogen;
n is 0 or 1;
R₆ is oxido;
with the proviso that if n=1, the N-atom bearing the radical R₆ has a positive charge;
R₇ is hydrogen or amino;
or a tautomer thereof, or a pharmaceutically acceptable salt, or a hydrate or solvate thereof.

The radicals and symbols as used in the definition of a compound of formula I have the meanings as disclosed in WO2006/122806 which publication is hereby incorporated into the present application by reference.

A compound of the present invention is a compound which is specifically described in WO2006/122806. A compound of the present invention is 2-methyl-2-[4-(3-methyl-2-oxo-8-quinolin-3-yl-2,3-dihydro-imidazo[4,5-c]quinolin-1-yl)-phenyl]-propionitrile and its monotosylate salt (COMPOUND A, also known as BEZ235). The synthesis of 2-methyl-2-[4-(3-methyl-2-oxo-8-quinolin-3-yl-2,3-dihydro-imidazo[4,5-c]quinolin-1-yl)-phenyl]-propionitrile is for instance described in WO2006/122806 as Example 7. Another compound of the present invention is 8-(6-methoxy-pyridin-3-yl)-3-methyl-1-(4-piperazin-1-yl-3-trifluoromethyl-phenyl)-1,3-dihydro-imidazo[4,5-c]quinolin-2-one (COMPOUND B). The synthesis of 8-(6-methoxy-pyridin-3-yl)-3-methyl-1-(4-piperazin-1-yl-3-trifluoromethyl-phenyl)-1,3-dihydro-imidazo[4,5-c]quinolin-2-one is for instance described in WO2006/122806 as Example 86. WO07/084786 describes pyrimidine derivatives, which have been found to inhibit the activity of lipid kinases, such as PI3-kinases. Specific pyrimidine derivatives which are suitable for the present invention, their preparation and suitable pharmaceutical formulations containing the same are described in WO07/084786 and include compounds of formula II

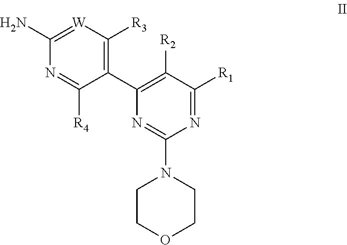

or a stereoisomer, tautomer, or pharmaceutically acceptable salt thereof, wherein,
W is CR_{w} or N, wherein R_{w} is selected from the group consisting of
(1) hydrogen,
(2) cyano,
(3) halogen,
(4) methyl,
(5) trifluoromethyl,
(6) sulfonamido;
R₁ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(22) —SO₂R₁ₐ, and
(23) —SO₂NR₁ₐR_{1b},
wherein R₁ₐ, and R_{1b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
R₂ is selected from the group consisting
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b},
wherein R₂ₐ, and R_{2b} are independently selected from the group consisting of
(a) hydrogen, and
(b) substituted or unsubstituted alkyl;
R₃ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(13) —NR₃ₐR_{3b},
(14) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, and
(20) —SO₂NR₃ₐR_{3b},
wherein R₃ₐ, and R_{3b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and
R₄ is selected from the group consisting of
(1) hydrogen, and
(2) halogen.

The radicals and symbols as used in the definition of a compound of formula II have the meanings as disclosed in WO07/084786 which publication is hereby incorporated into the present application by reference.

A compound of the present invention is a compound which is specifically described in WO07/084786. A compound of the present invention is 5-(2,6-di-morpholin-4-yl-pyrimidin-4-yl)-4-trifluoromethyl-pyridin-2-ylamine (COMPOUND C, also known as BKM120). The synthesis of 5-(2,6-di-morpholin-4-yl-pyrimidin-4-yl)-4-trifluoromethyl-pyridin-2-ylamine is described in WO07/084786 as Example 10.

In the context of the present invention, and as demonstrated in the examples, the PI3K inhibitor can be replaced by an inhibitor of the mammalian target of rapamycin (mTOR). Hence, as used herein, the terms “PI3K inhibitor” and “phosphoinositide 3-kinase (PI3K) inhibitor” compound also include mTOR inhibitors. In addition, as used herein, the terms “PI3K inhibitor” and “phosphoinositide 3-kinase (PI3K) inhibitor” also encompass inhibitors of other PI3K pathway components such as AKT. An mTOR inhibitor is a compound that decreases the activity of the target of rapamycin (mTOR) pathway. A decrease in activity of the target of rapamycin pathway is defined by a reduction of a biological function of the target of rapamycin. A target of rapamycin biological function includes for example, inhibition of the response to interleukin-2 (IL-2), blocking the activation of T- and B-cells, control of proliferation, and control of cell growth. An mTOR inhibitor acts for example by binding to protein FK-binding protein 12 (FKBP 12). mTOR inhibitors are known in the art or are identified using methods described herein. The mTOR inhibitor is for example a macrolide antibiotic such as rapamycin, temsirolimus (2,2-bis(hydroxymethyl)propionic acid; CCI-779), everolimus (RAD001) or ridaforolimus (AP23573) or mimetics or derivatives thereof. Further mTOR inhibitors are temsirolimus, ridaforolimus (also known as AP23573), MK-8669 (formerly known as Deforolimus), sirolimus, zotarolimus and biolimus. Mimetics and derivatives of rapamycin are known in the art such as those describes in U.S. Pat. Nos. RE37.421; 5,985,890; 5,912,253; 5,728,710; 5,712,129; 5,648,361; 7,332,601; 7,282,505; 6,680,330. Thus, as used herein, the term PI3K inhibitor also includes mTOR inhibitors and/or compounds which inhibit both PI3K and mTOR, e.g. Compound A.

Interleukin-8 (IL8), also known as interleukin 8, SCYB8, CXCL8, TSG-1, GCP-1, b-ENAP, MDNCF, OTTHUMP00000199824, MONAP, OTTHUMP00000199825, NAP-1, alveolar macrophage chemotactic factor I, GCP1, beta endothelial cell-derived neutrophil activating peptide, LECT, beta-thromboglobulin-like protein, LUCT, chemokine (C-X-C motif) ligand 8, LYNAP, emoctakin, NAF, NAP1, lung giant cell carcinoma-derived chemotactic protein, IL-8, lymphocyte derived neutrophil activating peptide, Granulocyte chemotactic protein 1, lymphocyte-derived neutrophil-activating factor, Monocyte-derived neutrophil chemotactic factor, neutrophil-activating peptide 1, Monocyte-derived neutrophil-activating peptide, small inducible cytokine subfamily B, member 8, C-X-C motif chemokine 8, T cell chemotactic factor, T-cell chemotactic factor, tumor necrosis factor-induced gene 1, 3-10C, Emoctakin, AMCF-I, Neutrophil-activating protein 1, K60, and Protein 3-10C is a chemokine produced by macrophages and other cell types such as epithelial cells. It is also synthesized by endothelial cells, which store IL-8 in their storage vesicles, the Weibel-Palade bodies. In humans, the interleukin-8 protein is encoded by the IL8 gene. There are several receptors on the surface membrane which are capable to bind IL-8; the most frequently studied types are the G protein-coupled serpentine receptors CXCR1, and CXCR2. Expression and affinity to IL-8 is different in the two receptors (CXCR1>CXCR2). Through a chain of biochemical reactions, IL-8 is secreted and is an important mediator of the immune reaction in the innate immune system response. Both monomer and homodimer forms of IL-8 were reported as potent inducers of CXCR1 and CXCR2. The homodimer proved to be more potent.

Chemokine (C-X-C motif) receptor 1, also known as CXCR1, interleukin 8 receptor, alpha, IL8RA, CD181 (cluster of differentiation 181), IL-8R A, CDw128a, C-C, C-C-CKR-1, CD181, CD128, CKR-1, IL8R1, interleukin 8 receptor, alpha, IL8RBA, High affinity interleukin-8 receptor A, OTTHUMP00000164140, CMKAR1, C-X-C chemokine receptor type 1, CXC-R1, interleukin-8 receptor type 1, CXCR-1, interleukin-8 receptor type A, IL-8 receptor type 1 and CD181 antigen is a chemokine receptor. It is a member of the G-protein-coupled receptor family. This protein is a receptor for interleukin 8 (IL8). It binds to IL8 with high affinity, and transduces the signal through a G-protein-activated second messenger system.

Inhibitors of the interaction of IL-8 and its receptors are well known in the art and have been described in e.g. WO1995/007934, WO1997/000893, WO1997/000601 and WO2002/077172.

Hence, the present invention also pertains to a combination such as a combined preparation or a pharmaceutical composition which comprises (a) a phosphoinositide 3-kinase (PI3K) inhibitor compound and (b) a compound which inhibits the interaction of IL-8 and its receptors. More particularly, in an embodiment, the present invention relates to a combination which comprises (a) a phosphoinositide 3-kinase (PI3K) inhibitor compound and (b) an antibody binding to IL-8 or to one of its receptors.

The terms “combination” and “combined preparation” as used herein also define a “kit of parts” in the sense that the combination partners (a) and (b) as defined above can be dosed independently or by use of different fixed combinations with distinguished amounts of the combination partners (a) and (b), i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the individual.

As shown in the examples, it has been found that combination therapy with a PI3K/mTOR inhibitor and different inhibitors of the interaction between IL-8 and its receptors results in unexpected improvement in the treatment of tumor diseases. When administered simultaneously, sequentially or separately, the PI3K/mTOR inhibitor and the compound which inhibits the interaction of IL-8 and its receptors interact in a synergistic manner to reduce cell number and tumor growth as well as decrease the number of circulating tumor cells and metastasis. This unexpected synergy allows a reduction in the dose required of each compound, leading to a reduction in the side effects and enhancement of the clinical effectiveness of the compounds and treatment.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients renders impractical the use of this form of testing as a primary model for synergy. However, the observation of synergy in one species can be predictive of the effect in other species and animal models exist, as described herein, to measure a synergistic effect and the results of such studies can also be used to predict effective dose and plasma concentration ratio ranges and the absolute doses and plasma concentrations required in other species by the application of pharmacokinetic/pharmacodynamic methods. Established correlations between tumor models and effects seen in man suggest that synergy in animals may e.g. be demonstrated in the tumor models as described in the Examples below.

In one aspect the present invention provides a synergistic combination for human administration comprising (a) PI3K inhibitor compound and (b) a compound which inhibits the interaction of IL-8 and its receptors, or pharmaceutically acceptable salts or solvates thereof, in a combination range (w/w) which corresponds to the ranges observed in a tumor model, e.g. as described in the Examples below, used to identify a synergistic interaction. Suitably, the ratio range in humans corresponds to a non-human range selected from between 50:1 to 1:50 parts by weight, 50:1 to 1:20, 50:1 to 1:10, 50:1 to 1:1, 20:1 to 1:50, 20:1 to 1:20, 20:1 to 1:10, 20:1 to 1:1, 10:1 to 1:50, 10:1 to 1:20, 10:1 to 1:10, 10:1 to 1:1, 1:1 to 1:50, 1.1 to 1:20 and 1:1 to 1:10. More suitably, the human range corresponds to a non-human range of the order of 10:1 to 1:1 or 5:1 to 1:1 or 2:1 to 1:1 parts by weight.

According to a further aspect, the present invention provides a synergistic combination for administration to humans comprising an (a) a PI3K inhibitor compound and (b) a compound which inhibits the interaction of IL-8 and its receptors or pharmaceutically acceptable salts thereof, where the dose range of each component corresponds to the synergistic ranges observed in a suitable tumor model, e.g. the tumor models described in the Examples below, primarily used to identify a synergistic interaction. Suitably, the dose range of the PI3K inhibitor compound in human corresponds to a dose range of 1-1000 mg/kg, for instance 1-500 mg/kg, 1-200 mg/kg, 1-100 mg/kg, 1-50 mg/kg, 1-30 mg/kg (e.g. 1-35 mg/kg or 1-10 mg/kg for Compound A, 1-25 mg/kg for Compound B) in a suitable tumor model, e.g. a mouse model as described in the Examples below.

For the compound which inhibits the interaction of IL-8 and its receptors, the dose range in the human suitably corresponds to a synergistic range of 1-50 mg/kg or 1-30 mg/kg (e.g. 1-25 mg/kg, 1-10 mg/kg or 1-2.5 mg/kg) in a suitable tumor model, e.g. a mouse model as described in the Examples below. Suitably, the dose of PI3K inhibitor compound for use in a human is in a range selected from 1-1200 mg, 1-500 mg, 1-100 mg, 1-50 mg, 1-25 mg, 500-1200 mg, 100-1200 mg, 100-500 mg, 50-1200 mg, 50-500 mg, or 50-100 mg, suitably 50-100 mg, once daily or twice daily (b.i.d.) or three times per day (t.i.d.), and the dose of the compound which inhibits the interaction of IL-8 and its receptors is in a range selected from 1-1000 mg, 1-500 mg, 1-200 mg, 1-100 mg, 1-50 mg, 1-25 mg, 10-100 mg, 10-200 mg, 50-200 mg or 100-500 mg once daily, b.i.d or t.i.d.

In accordance with a further aspect the present invention provides a synergistic combination for administration to humans comprising an (a) a PI3K inhibitor compound at 10%-100%, preferably 50%-100% or more preferably 70%-100%, 80%-100% or 90%-100% of the maximal tolerable dose (MTD) and (b) a compound which inhibits the interaction of IL-8 and its receptors at 10%-100%, preferably 50%-100% or more preferably 70%-100%, 80%-100% or 90%-100% of the MTD. In an embodiment one of the compounds, e.g. the PI3K inhibitor compound, is dosed at the MTD and the other compound, e.g. the compound which inhibits the interaction of IL-8 and its receptors, is dosed at 50%-100% of the MTD, preferably at 60%-90% of the MTD. The MTD corresponds to the highest dose of a medicine that can be given without unacceptable side effects. It is within the art to determine the MTD. For instance the MTD can suitably be determined in a Phase I study including a dose escalation to characterize dose limiting toxicities and determination of biologically active tolerated dose level.

In one embodiment of the invention, (a) the phosphoinositide 3-kinase (PI3K) inhibitor compound inhibitor is selected from the group consisting of COMPOUND A, COMPOUND B or COMPOUND C. In one embodiment of the invention, (b) the compound which inhibits the interaction of IL-8 and its receptors is an antibody specifically binding to either CXCR1, CXCR2 or IL-8.

The term “treating” or “treatment” as used herein comprises a treatment affecting a delay of progression of a disease. The term “delay of progression” as used herein means administration of the combination to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

The subject to be treated is usually a human. Although mostly referring to human, the present invention is however not limited to human. In the present invention, the subject can be any warm-blooded animal, including, next to human, but not limited to, animals such as cows, pigs, horses, chickens, cats, dogs, camels, etc.

In one embodiment of the present invention, the proliferative disease is breast cancer, in particular a metastatic breast cancer or a breast cancer of the triple negative type.

In another embodiment of the present invention, the proliferative disease is a solid tumor. The term “solid tumor” especially means breast cancer, ovarian cancer, cancer of the colon and generally the GI (gastro-intestinal) tract, cervix cancer, lung cancer, in particular small-cell lung cancer, and non-small-cell lung cancer, head and neck cancer, bladder cancer, cancer of the prostate or Kaposi's sarcoma. The present combination inhibits the growth of solid tumors, but also liquid tumors. Furthermore, depending on the tumor type and the particular combination used a decrease of the tumor volume can be obtained. The combinations disclosed herein are also suited to prevent the metastatic spread of tumors, e.g. of breast cancer, and the growth or development of micrometastases. The combinations disclosed herein are in particular suitable for the treatment of poor prognosis patients.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

It will be understood that references to the combination partners (a) and (b) are meant to also include the pharmaceutically acceptable salts. If these combination partners (a) and (b) have, for example, at least one basic center, they can form acid addition salts. Corresponding acid addition salts can also be formed having, if desired, an additionally present basic center. The combination partners (a) and (b) having an acid group (for example COOH) can also form salts with bases. The combination partner (a) or (b) or a pharmaceutically acceptable salt thereof may also be used in form of a hydrate or include other solvents used for crystallization.

A combination which comprises (a) a phosphoinositide 3-kinase inhibitor compound and (b) a compound which inhibits the interaction of IL-8 and its receptors, in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATION OF THE INVENTION has both synergistic and additive advantages, both for efficacy and safety. Therapeutic effects of combinations of a phosphoinositide 3-kinase inhibitor compound with a compound which inhibits the interaction of IL-8 and its receptors can result in lower safe dosages ranges of each component in the combination.

The pharmacological activity of a COMBINATION OF THE INVENTION may, for example, be demonstrated in a clinical study or in a test procedure as essentially described hereinafter. Suitable clinical studies are, for example, open label non-randomized, dose escalation studies in patients with advanced solid tumors. Such studies can prove the additive or synergism of the active ingredients of the COMBINATIONS OF THE INVENTION. The beneficial effects on proliferative diseases can be determined directly through the results of these studies or by changes in the study design which are known as such to a person skilled in the art. Such studies are, in particular, suitable to compare the effects of a monotherapy using the active ingredients and a COMBINATION OF THE INVENTION. Preferably, the combination partner (a) is administered with a fixed dose and the dose of the combination partner (b) is escalated until the Maximum Tolerated Dosage (MTD) is reached.

It is one objective of this invention to provide a pharmaceutical composition comprising a quantity, which is therapeutically effective against a proliferative disease comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including man. Alternatively, when the agents are administered separately, one can be an enteral formulation and the other can be administered parenterally.

The novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, for example, water, glycols, oils, alcohols, flavoring agents, preservatives, coloring agents; or carriers such as starches, sugars, microcristalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

In particular, a therapeutically effective amount of each of the combination partner of the COMBINATION OF THE INVENTION may be administered simultaneously or sequentially and in any order, and the components may be administered separately or as a fixed combination. For example, the method of delay of progression or treatment of a proliferative disease according to the invention may comprise (i) administration of the first combination partner in free or pharmaceutically acceptable salt form and (ii) administration of the second combination partner in free or pharmaceutically acceptable salt form, simultaneously or sequentially in any order, in jointly therapeutically effective amounts, preferably in synergistically effective amounts. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The COMBINATION OF THE INVENTION can be a combined preparation or a pharmaceutical composition.

Moreover, the present invention relates to a method of treating a warm-blooded animal having a proliferative disease comprising administering to the animal a COMBINATION OF THE INVENTION in a quantity which is therapeutically effective against said proliferative disease.

Furthermore, the present invention pertains to the use of a COMBINATION OF THE INVENTION for the treatment of a proliferative disease and for the preparation of a medicament for the treatment of a proliferative disease.

Moreover, the present invention provides a commercial package comprising as active ingredients COMBINATION OF THE INVENTION, together with instructions for simultaneous, separate or sequential use thereof in the delay of progression or treatment of a proliferative disease. Embodiments of the invention are represented by combinations comprising
An antibody specifically binding to IL-8 and one or more compound selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.
An antibody specifically binding to CXCR1 and one or more compound selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.
An antibody specifically binding to CXCR2 and one or more compound selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.
Repertaxin (also known as reparixin) and one or more compound selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.
A siRNA decreasing the expression of IL-8 and one or more compound selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.
A siRNA decreasing the expression of CXCR1 and one or more compound selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.
A siRNA decreasing the expression of CXCR2 and one or more compound selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.

In another embodiment, the invention provides combinations comprising
COMPOUND A and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
COMPOUND B and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
COMPOUND C and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
Rapamycin and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
Temsirolimus and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
Everolimus and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
Temsirolimus and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
Ridaforolimus and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
MK-8669 and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
Sirolimus and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
Zotarolimus and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.
Biolimus and one or more compound selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.

In further aspects, the present inventions provides
a combination which comprises (a) a COMBINATION OF THE INVENTION, wherein the active ingredients are present in each case in free form or in the form of a pharmaceutically acceptable salt or any hydrate thereof, and optionally at least one pharmaceutically acceptable carrier; for simultaneous, separate or sequential use;
a pharmaceutical composition comprising a quantity which is jointly therapeutically effective against a proliferative disease of a COMBINATION OF THE INVENTION and at least one pharmaceutically acceptable carrier;
the use of a COMBINATION OF THE INVENTION for the treatment of a proliferative disease;
the use of a COMBINATION OF THE INVENTION for the preparation of a medicament for the treatment of a proliferative disease;
the use of a combination COMBINATION OF THE INVENTION wherein the PI3K inhibitor is selected from COMPOUND A, COMPOUND B, COMPOUND C, rapamycin, temsirolimus, everolimus, temsirolimus, ridaforolimus, MK-8669 sirolimus, zotarolimus and biolimus; and
the use of a COMBINATION OF THE INVENTION wherein the compound which inhibits the interaction between IL-8 and its receptors is selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.

Moreover, in particular, the present invention relates to a combined preparation, which comprises (a) one or more unit dosage forms of a phosphoinositide 3-kinase inhibitor compound and (b) a compound which inhibits the interaction between IL-8 and its receptors.

Furthermore, in particular, the present invention pertains to the use of a combination comprising (a) a phosphoinositide 3-kinase inhibitor compound and (b) a compound which inhibits the interaction between IL-8 and its receptors for the preparation of a medicament for the treatment of a proliferative disease.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

COMPOUND A may be administered to a human in a dosage range varying from about 50 to 1000 mg/day. COMPOUND B may be administered to a human in a dosage range varying from about 25 to 800 mg/day. COMPOUND C may be administered to a human in a dosage range varying from about 25 to 800 mg/day.

As demonstrated in the examples, the term “compound” as used herein also includes siRNA decreasing or silencing the expression of a target gene. “RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown’ of gene expression. “siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesized de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridizes to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid. The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA. Frequently, automatically generated alignments require manual alignment, exploiting the trained user's knowledge of the protein family being studied, e.g., biological knowledge of key conserved sites. One such alignment editor programs is Align (http://www.gwdg.de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable. Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYL1P. The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In an embodiment, the inhibitor is a siRNA molecule and comprises between approximately 5 bp and 50 bp, in some embodiments, between 10 bp and 35 bp, or between 15 bp and 30 bp, for instance between 18 bp and 25 bp. In some embodiments, the siRNA molecule comprises more than 20 and less than 23 bp.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides. The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among: a) a 3′ cap; b) a 5′ cap, c) a modified internucleoside linkage; or d) a modified sugar or base moiety. Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates. End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analyzing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference. The siRNA molecule may be either synthesized de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918. The reaction may be carried out in solution or, in some embodiments, on solid phase or by using polymer supported reagents, followed by combining the synthesized RNA strands under conditions, wherein a siRNA molecule is formed, which is capable of mediating RNAi. It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used. For the avoidance of doubt, the term siRNA as used herein also includes miRNA, shRNA and shRNAmir.

Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators). The gene-silencing molecule may be either synthesized de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell. The molecule may be produced by a vector harboring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesize the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favor targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like). Alternatively a “naked” siRNA or antisense molecule may be inserted into a subject's cells by a suitable means e.g. direct endocytotic uptake.

The gene silencing molecule may also be transferred to the cells of a subject to be treated by transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly.

In an embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product).

As demonstrated in the examples, the term “compound” as used herein also includes antibodies. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgGI, IgG2, IgG3, IgG4, IgA1 and IgA2) or subclass of immunoglobulin molecule.

In addition, in the context of the present invention, the term “antibody” shall also encompass alternative molecules having the same function of specifically recognizing proteins, e.g. aptamers and/or CDRs grafted onto alternative peptidic or non-peptidic frames.

In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CHI, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. In some embodiments, the antibodies are human, murine (e.g., mouse and rat), donkey, sheep, rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992). Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues.

Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide. Antibodies may act as agonists or antagonists of the recognized polypeptides. The invention also features receptor-specific antibodies which do not prevent ligand binding but prevent receptor activation. Receptor activation (i.e., signalling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by Western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387.

The antibodies as defined for the present invention include derivatives that are modified, i. e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821, 047; 5,571, 698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e. g., PCT publications WO 98/24893; WO 92/01047; WO 96/34096; WO 96/33735; European Patent No. 0 598 877; U.S. Pat. Nos. 5,413,923; 5,625,126; 5,633,425; 5,569, 825; 5,661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Biol/technology 12:899-903 (1988)).

Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity. Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)). The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide). Further, in some embodiment of the invention an antibody, or fragment thereof, recognizing specifically IL8 and/or CXCR1 may be conjugated to a therapeutic moiety. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et al, Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980.

The present invention is also directed to antibody-based therapies which involve administering antibodies of the invention to an animal, in some embodiments, a mammal, for example a human, patient to treat cancer. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

As used herein, the term “metastasis” refers to the spread of cancer cells from one organ or body part to another area of the body, i.e. to the formation of metastases. This movement of tumor growth, i.e. metastasis or the formation of metastases, occurs as cancer cells disseminate from the original tumor and spread e.g. by way of the blood or lymph system. Without wishing to be bound by theory, metastasis is an active process and involves an active breaking from the original tumor, for instance by protease digestion of membranes and or cellular matrices, transport to another site of the body, for instance in the blood circulation or in the lymphatic system, and active implantation at said other area of the body.

The following Examples illustrate the invention described above; they are not, however, intended to limit the scope of the invention in any way. The beneficial effects of the COMBINATION OF THE INVENTION can also be determined by other test models known as such to the person skilled in the pertinent art.

### FIGURE LEGEND

FIG. 1. Dual PI3K/mTOR inhibition increases, while JAK2 inhibition blocks IL-8 secretion (A) Bar graphs showing IL-8 levels measured by ELISA (left and middle) or quantification of dots of cytokine arrays (right) from tumors of mice treated with vehicle control (VHC), 30 mg/kg BEZ235 (BEZ), 120 mg/kg NVP-BSK805 (BSK), or 25 mg/kg BEZ and 100 mg/kg BSK. For MDA231 LM2 shJAK2 tumors, JAK2 was inhibited by doxycycline (dox) administration, leading to activation of the JAK2 shRNA (shJAK2). shNT refers to non-targeting shRNA. Results represent the means±SEM (n=3-8, *P<0.05). (B) Bar graphs showing IL-8 levels measured by ELISA in plasma of mice bearing tumors treated as in FIG. 1A. Results represent means±SEM (n=4, *P<0.05).

FIG. 2. Blockade of CXCR1 reduces invasion and has minor effects on primary tumor growth (A) NVP-BSK805 and CXCR1 blockade but not BEZ235 decrease invasion. Bar graphs showing relative invasion of MDA231 LM2 cells seeded on Matrigel coated Boyden invasion chambers and treated with 300 nM BEZ235, 350 nM NVP-BSK805, a JKA2 inhibitor, and/or CXCR1 blocking antibody. Invasion was assessed after 48 h, data represent relative invasion values normalized to cell number and are means±SEM (n=4, *P<0.05).

(B) Effects of inhibition of IL-8 signaling in vivo by Repertaxin on primary tumor growth. Growth curves of tumors from mice treated with vehicle control (VHC), 30 mg/kg BEZ235, 120 mg/kg NVP-BSK805, 30 mg/kg Repertaxin or 25 mg/kg BEZ235, 100 mg/kg NVP-BSK805 and 30 mg/kg Repertaxin. Injection refers to orthotopic cell injection and the arrows indicate initiation of treatment. Results are presented as mean tumor volume±SEM (n=4-8, *P<0.05).

FIG. 3. Inhibition of IL-8 signaling by CXCR1 blockade or JAK2 inhibition blocks Circulating Tumor Cells (CTCs) and metastatic indices. Bar graphs showing CTC (left panel) and lung metastatic (right panel) indices of mice treated as in FIG. 2B. Results are presented as means±SEM (n=3-4, *P<0.05).

FIG. 4. BEZ235 treatment activates JAK2/STAT5 and IL-8 secretion in human primary triple-negative breast tumors Immunoblots of lysates from primary triple-negative breast tumors grown in immunodeficient mice and treated for 4 days with 30 mg/kg BEZ235 or vehicle (VHC). pJAK2 levels were measured in triplicate by ELISA and normalized to total JAK2 levels (Y-axis).

FIG. 5. BEZ235 treatment activates JAK2/STAT5 and IL-8 secretion in human primary triple-negative breast tumors Bar graphs showing IL-8 levels measured by ELISA in the dissected tumors from or in the plasma of mice at day 3 of treatment with 30 mg/kg BEZ235 or vehicle (VHC). Results represent means±SEM (n=3-4, *P<0.05).

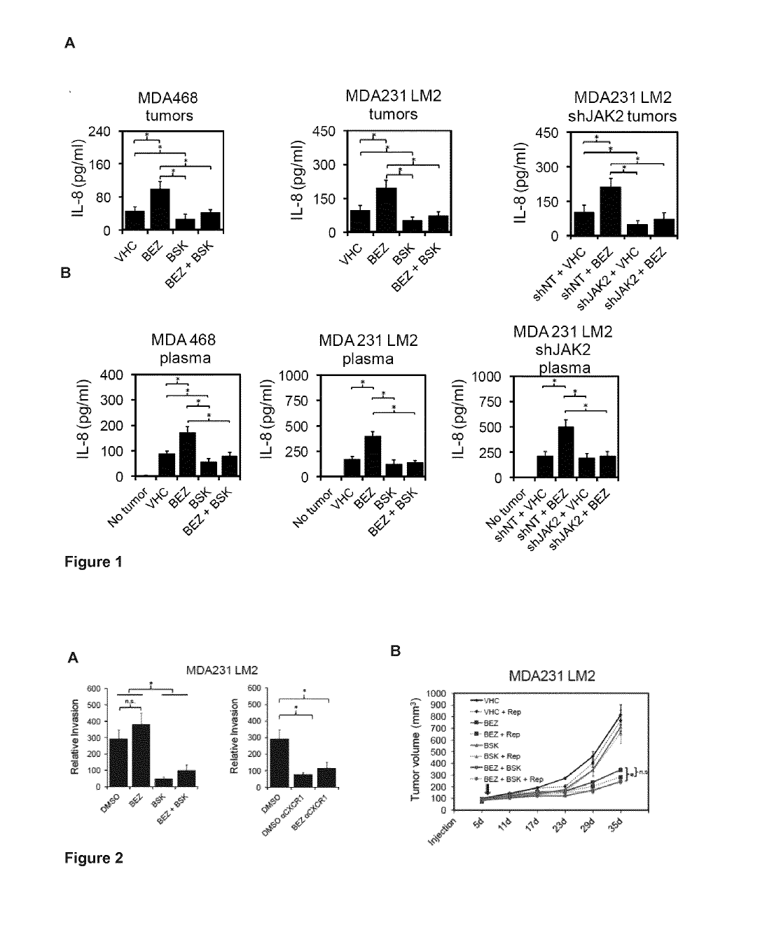

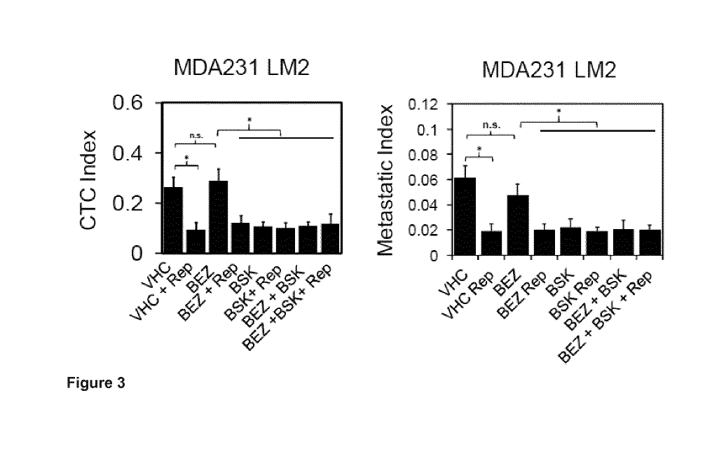

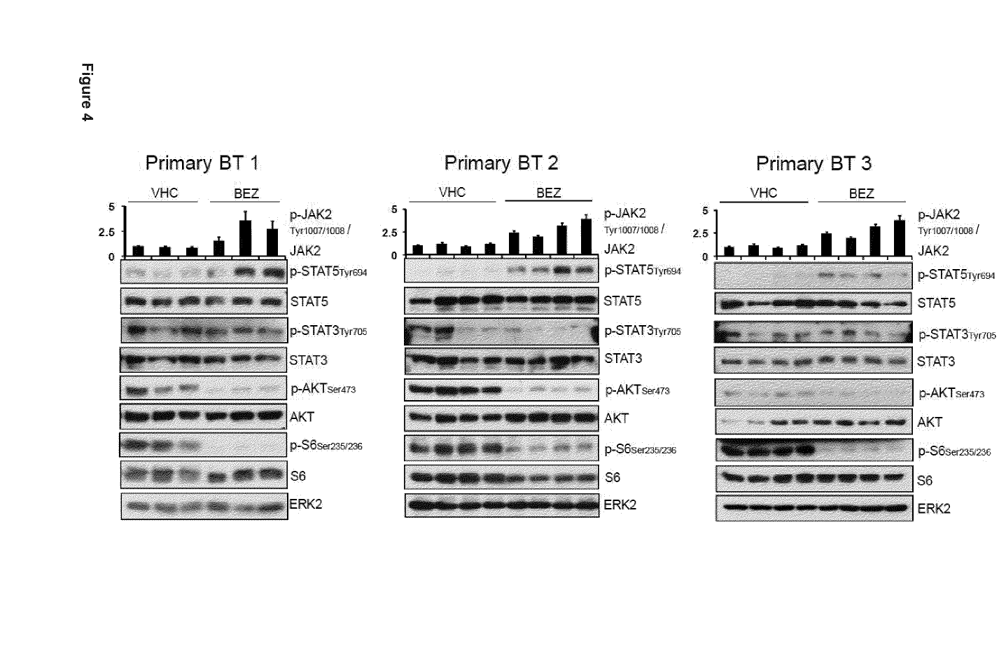

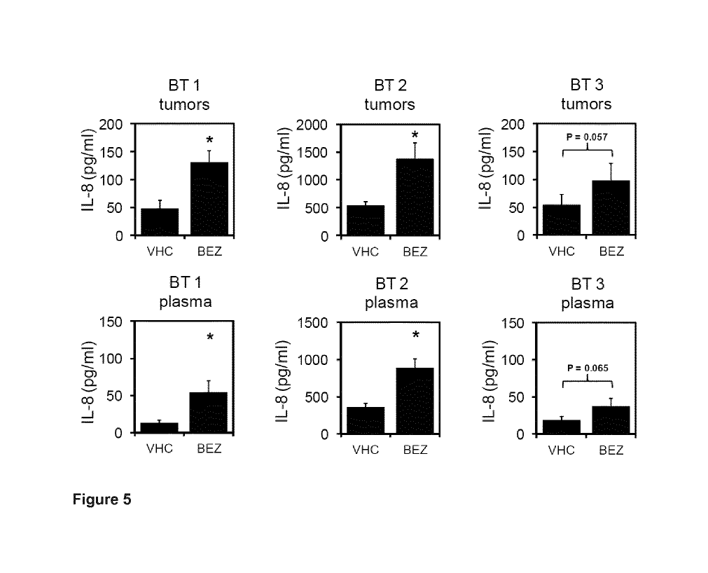

### EXAMPLES

### Compounds and Formulations

BEZ235 (AN4) (PI3K/mTOR inhibitor), NVP-BSK805 (JAK2 inhibitor), NVP-BKM120 (pan-PI3K inhibitor) and RAD001 (mTORC1 inhibitor) were all from Novartis, Basel, Switzerland. Repertaxin L-lysine salt was obtained from WuXi AppTec Co., Ltd (Shanghai, China). Compounds were prepared as 10 mmol/L stock solutions in DMSO and stored protected from light at −20° C. For dosing of mice, NVP-BSK805 was freshly formulated in NMP/PEG300/Solutol HS15 (5%/80%/15%), BEZ235 was freshly formulated in NMP/PEG300 (10%/90%) and both were applied at 10 mL/kg by oral gavage. Repertaxin was freshly formulated in PBS and administered s.c. at 20 mg/kg.

### Cell Lines, Cell Culture and In Vitro Experiments

MDA231, lung metastatic subline MDA231 LM2 (Minn et al., 2005) and SUM159 human breast cancer cells, as well as the Balb/c tumor-derived mammary cancer lines 4T-1 and 168FARN (Aslakson and Miller, 1992), were propagated as previously described. All other cell lines were obtained from and were cultured according to the protocols of ATCC. For treatment with inhibitors, cells were synchronized without serum overnight and then supplemented with culture medium containing the inhibitor(s). For experiments with doxycycline-inducible shRNAs, 500 ng/ml of doxycycline (Sigma) was added to the medium and experiments performed 48 h later. Cell viability assays, cell cycle and cell death measurements were performed at 0.5% FCS in order to avoid masking effects of growth factors present under full-serum conditions.

Cytokine stimulation of cells was performed for 30 min in medium with 0.5% FCS supplemented with cytokines at 10 ng/ml, except for EPO at 10 U/ml. Antibody-blocking experiments were performed by adding anti-CXCR1 (R&D, MAB330, 1 μg/ml), anti-CXCR2 (R&D, MAB331, 2.5 μg/ml) or a mouse IgG antibody (R&D, 1 μg/ml) to the medium 45 min prior to lysis of the cells. Cell viability was measured using the Cell Proliferation Reagent WST-1 (Roche). Colony formation assays were performed by seeding 1000 cells/well in 6-well plates and staining single colonies after 7-14 days with 0.2% crystal violet in PBS/4% formalin. Matrigel invasion assays were performed using BD BioCoat Matrigel Invasion Chambers according to the manufacture's protocol (BD Bioscience). The number of invading cells in each treatment condition was counted 48 h after seeding by microscopy at 40× (using the mean of 4 microscopic fields) and normalized to cell number.

### Immunoblotting and Immunoprecipitation

Cells for Western Blotting and ELISA were lysed with RIPA buffer. Xenograft lysates were prepared by lysing kryo-homogenized tumor powder in RIPA buffer (50 mM Tris-HCl pH 8, 150 mM NaCl, 1% NP-40, 0.5% sodium deoxycholate, 0.1% SDS. RIPA was supplemented with 1× protease inhibitor cocktail (Complete Mini, Roche), 0.2 mmol/L sodium-vanadate, 20 mM sodium fluoride and 1 mmol/L phenylmethylsulfonyl fluoride. For IRS-1 immunoprecipitation, cell lysates containing 500-1000 μg of protein were incubated with 1 μg of antibody and 20-50 μl of protein A-Sepharose beads (Zymed Laboratories, Inc., South San Francisco, Calif.) overnight at 4° C. Immunoprecipitates or whole cell lysates (30-80 μg) were subjected to SDS-PAGE, transferred to PVDF membranes (Immobilon-P, Millipore) and blocked for 1 hr at room temperature with 5% milk in PBS-0.1% Tween 20. Membranes were then incubated overnight with antibodies as indicated and exposed to secondary HRP-coupled anti-mouse or -rabbit antibody at 1:5-10,000 for 1 h at room temperature. Proteins were visualized using an ECL kit (Amersham) or an enhanced chemiluminescence detection system (Pierce Biotechnology). In each of the studies presented, the results shown are typical of at least three independent experiments. The following antibodies were used: anti-JAK2 (Cell Signaling), anti-JAK1 (Cell Signaling), anti-pSTAT5 (Tyr694, Cell Signaling), anti-STAT5 (STAT5A&B, Cell Signaling), anti-STAT3 (Cell Signaling), anti-pSTAT3 (Tyr705, Cell Signaling), anti-AKT pan (Cell Signaling), anti-pAKT (Thr308 and Ser473, Cell Signaling), anti-ERK2 (Santa Cruz), anti-S6 (Cell Signaling), anti-pS6 (Ser235/236, Cell Signaling), anti-PARP (Cell Signaling), anti-MCL1 (Cell Signaling), anti-BIM (EL, L and S isoforms, Cell Signaling), anti-pIGF1R/plnsR (Invitrogen), anti-IGF1Rbeta (Cell Signaling), anti-InsRbeta (Santa Cruz), anti-IRS1 (Upstate), anti-plRS1 (Tyr612, Calbiochem).

### ELISA and Cytokine Arrays

For assessing pJAK2 levels, an ELISA assay (Tyr1007/1008, Invitrogen) was applied because of cross-reactivity of all pJAK2 antibodies tested. Interleukin-8 levels in RIPA lysates, cell culture supernatants and mouse tail vein blood plasma were measured by ELISA, as well (Biolegend). Cytokine arrays on cell culture supernatants and mouse tumor lysates were performed according the manufacture's protocol (R & D systems, Human and Mouse cytokine array panel A).

### RNA Preparation and RQ-PCR

Total RNA was extracted using the RNeasy Mini Kit and DNase elimination columns according to the manufacturer's protocol (Qiagen). 1 μg of total RNA were transcribed using the Thermo Script RT-PCR System from Invitrogen. PCR and fluorescence detection were performed using the StepOnePlus Sequence Detection System (Applied Biosystems, Rotkreuz, Switzerland) according to the manufacturer's protocol in a reaction volume of 20 μl containing 1× TaqMan® Universal PCR Master Mix (Applied Biosystems) and 25 ng cDNA. For quantification of IL-8, GAPDH and RPLPO mRNA, the 1× Taqman® Gene Expression Assays Hs00174103_m1, Hs02758991_g1 and Hs99999902_m1 (Applied Biosystems) were used. All measurements were performed in duplicates and the arithmetic mean of the Ct-values was used for calculations: target gene mean Ct-values were normalized to the respective housekeeping genes (GAPDH and RPSO), mean Ct-values (internal reference gene, Ct), and then to the experimental control. Obtained values were exponentiated 2(−ΔΔCt) to be expressed as n-fold changes in regulation compared to the experimental control (2(−ΔΔCt) method of relative quantification (Livak and Schmittgen, 2001).

### Gene Silencing Procedures

siRNAs were ordered as RP-HPLC purified duplexes from Sigma-Aldrich, the sequences were the following: siJAK1_{—}1 5′-GCACAGAAGACGGAGGAAAUGGUAU-3′ (SEQ ID NO:1), siJAK1_{—}2 5′-GCCUUAAGGAAUAUCUUCCAAAGAA-3′ (SEQ ID NO:2), si-IRS1:5′-AACAAGACAGCUGGUACCAGG-3′ (SEQ ID NO:3), siNT (non-targeting control) 5′-AUUCUAUCACUAGCGUGACUU-3′ (SEQ ID NO:4). For JAK2, Validated Stealth RNAi™ siRNA were ordered from Sigma-Aldrich (VHS41246). Transfections of siRNAs were performed using according to the manufacture's guidelines (Dharma Fect 1, Dharmacon). For lentiviral production, 293T cells were plated at a density of 2.5×10⁶ cells per 10 cm culture dish. Cells were cotransfected by PEI method (PEI:DNA ratio=4:1) with either 15 μg of pLKO1-tet-on-JAK2 shRNA (#629, target sequence: TGGATAGTTACAACTCGGCTT (SEQ ID NO:5)) or pLK01-tet-on-non-silencing shRNA (Wiederschain et al., 2009) and 10 μg of 3^{rd} generation packaging plasmid mix. The culture medium was replaced with fresh medium after 16 hr. Supernatant was collected 48 and 72 hr after transfection. For determining the viral titers, 10⁵ MDA-MB-468 and MDA-MB-231-LM2 cells were seeded in a six-well plate and transduced with various dilutions of the vector in the presence of 8μ of Polybrene per milliliter (Sigma-Aldrich). The culture medium was replaced 72 hr later with fresh medium containing puromycin (Sigma-Aldrich) at a concentration of 1.5 μg/ml. MDA-MD-468 and MDA-MB-231-LM2 cells transduced with viral vector at a multiplicity of infection of 20 were used for experiments.

### Flow Cytometry

Cells were detached using Trypsin-EDTA, resuspended in normal growth medium and counted. Tumors were mechanically and enzymatically dissociated (using collagenase II and HyQtase digestion). For Annexin V staining, 0.5×10⁶ cells were washed with cold PBS/5% BSA, resuspended in 70 μl binding buffer and labelled with phycoerythrin (PE)-labelled antibody against Annexin V according to the manufacturer's protocol (Becton Dickinson). For cell cycle analysis, 1×10⁶ cells were washed in PBS, fixed in 70% Ethanol for 60 min at 4° C., washed twice and resuspended in PI buffer (PBS supplemented with 50 μg/ml propidium iodide, 10 μg/ml RNAse A, 0.1% sodium citrate and 0.1% Triton X-100). For analysis of CXCR1 and CXCR2 cell surface expression, cells were incubated with 2.5 μg/10⁶ cells anti-CXCR1 (R&D, MAB330), anti-CXCR2 (R&D, MAB331) or with 1 μg/10⁶ cells mouse IgG antibody (R&D) for 20 min at 4° C., then with a secondary anti-mouse IgG-AlexaFluor647 (Biolegend) for 15 min at 4° C. in the dark prior to washing and analysis. At least 10⁴ cells per sample were analyzed with a FACScan flow cytometer (Becton Dickinson, Basel, Switzerland).

### Animal Experiments.

SCID/beige, SCID/NOD and Balb/c mice (Jackson Labs) were maintained under specific pathogen-free conditions and were used in compliance with protocols approved by the Institutional Animal Care and Use Committees of the FMI, which conform to institutional and national regulatory standards on experimental animal usage. For orthotopical engraftment of breast cancer cell lines, 1×10⁶ MDA-MB-468, 1×10⁶ MDA-MB-231-LM2 and 0.5×10⁶ 4 T-1 or 4T-1-GFP cells were suspended in a 100-μl mixture of Basement Membrane Matrix Phenol Red-free (BD Biosciences) and PBS 1:1 and injected into the mammary gland 4 or between mammary glands 2 and 3. Primary patient breast tumors were cut into 1 mm×1 mm pieces and transplanted into mammary gland 4. Tumor-bearing mice were randomized based on tumor volume prior to the initiation of treatment, which was initiated when average tumor volume was at least 100 mm³. BEZ-235 and BKS-805 were given orally (formulations see above) on each of 6 consecutive days followed by one day without the drug. Repertaxin was administered at 20 mg/kg s.c. daily. Expression of shRNAs was induced by adding doxycycline in the drinking water (2 g/l of in a 5% sucrose solution), which was refreshed every 48 h. Tumors were measured every 3 to 4 days with vernier calipers, and tumor volumes were calculated by the formula 0.5×(larger diameter)×(smaller diameter)². End point tumor sizes were analyzed for synergism using the formula AB/C<A/C×B/C, where C=tumor volume VHC, A=tumor volume compound 1, B=tumor volume compound 2, AB=tumor volume combination (Clarke, 1997).

### Immunohistochemistry.

Tumors were fixed in 10% NBF (Neutral buffered formalin) for 24 h at 4° C., washed with 70% EtOH, embedded in paraffin and stained with H&E, anti-Ki67 (Thermo Scientific), anti-pSTAT5 (Tyr694, cell signaling), anti-pAKT (Ser473, cell signaling), anti-pS6 (Ser235/236, cell signaling), anti-PARP (cell signaling) and anti-mouse F4/80 (AbD Serotec) antibodies. Mouse lungs were fixed in Bouin's fixative and visible metastatic lung nodules were counted using a binocular.

### Statistical Analysis.

Each value reported represents the mean±S.E.M. of at least three independent experiments. Data were tested for normal distribution and Student's t-test, ANOVAs or nonparametric Mann-Whitney U/Wilcoxon-tests were applied. To account for multiple comparisons, Tukey HSD and Wilcoxon tests were performed. The programs JMP4 and JMP9 were used for all statistical tests (SAS, Cary, N.C., USA). P values <0.05 were considered to be statistically significant. For the calculation of tumor initiating cell frequency, estimates and confidence intervals were calculated using R and the “statmod” package (Hu and Smyth, 2009) based on the method by (Shackleton et al., 2006).

The present inventors applied single doses of COMPOUND A, a dual PI3K and mTOR inhibitor, and analyzed target inhibition and potential signaling pathway crosstalks after 2, 4, 8 and 20 h hours of treatment. They found that COMPOUND A reduced pAKT and completely blocked pS6 levels up to 20 hours after treatment in the PTEN-deficient MDA 468 and the RAS-mutated MDA 231 LM2 breast cancer lines, as well as in the mouse breast cancer line 4T-1. The present inventors further used in vivo models to confirm these results. Surprisingly, they detected a considerable upregulation of pJAK2 and pSTAT5 after 4 hours-8 hours of BEZ235 treatment in vitro and after 8 hours of treatment in vivo. Levels of pSTAT3 remained however largely unaffected by BEZ235 treatment. In order to elucidate which arm of the dual inhibitor COMPOUND A could be responsible for the observed crosstalk to JAK2, the present inventors used a PI3K-specific inhibitor (BKM120) and an mTOR inhibitor (RAD001). They found that both single inhibition of PI3K and mTOR upregulated pJAK2 and pSTAT5, however at different time points. While RAD001 readily activated JAK2 starting at 4 hours of treatment, they observed a later response with BKM120 treatment starting at 8 hours after adding the compound. Given the fact that both JAK2 and JAK1 are capable of signaling to STAT5 and STAT3 depending on the cell type and the receptor they are associated with (Desrivieres et al., 2006; Bezbradica et al., 2009), the present inventors performed siRNA depletion of both JAKs and found that only JAK2 is responsible for activation of STAT5 while JAK1 is upstream of STAT3 in the experimental models used. Next, they investigated whether JAK2 activation is necessary for upregulation of pSTAT5 by BEZ235 treatment and if a highly specific JAK2 inhibitor, COUMPOUND D (Baffert et al, 2010), would be sufficient to block this crosstalk. The results show that both siRNA depletion of JAK2 and inhibition of its activity counteracted upregulation of pSTAT5 by BEZ235. Hence, the inventors found a JAK2/STAT5-evoked positive feedback loop that causes resistance to dual PI3K/mTOR inhibition. Mechanistically, PI3K/mTOR inhibition increased IRS1-dependent activation of JAK2/STAT5 and secretion of IL-8 in several cell lines and primary triple-negative breast cancer. Genetic or pharmacological inhibition of JAK2 abrogated this feedback loop. They further showed that combined PI3K/mTOR and JAK2 inhibition synergistically reduced cancer cell number in vitro, as well as tumor growth, the number of circulating tumor cells and metastasis in vivo. The inventors' study thus revealed a new link between growth factor signaling, JAK/STAT activation and cytokine secretion. Their results provide a rationale for combined targeting of the PI3K/mTOR and JAK2/STAT5 pathways in proliferative diseases.

TABLE 1
BEZ increased phosphorylation of JAK2/STAT5 and IL-8
secretion in a panel of breast cancer cell lines.

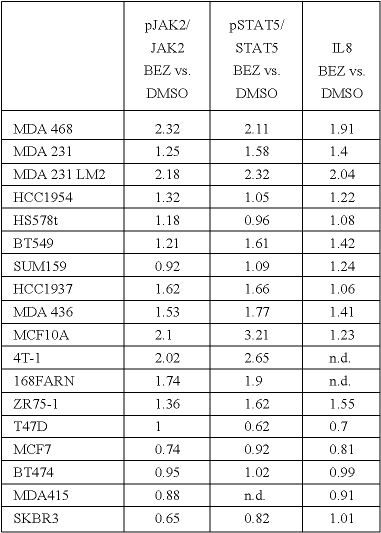

Shown are the levels of JAK2/STAT5 phosphorylation and IL-8 secretion upon treatment of triple-negative (bold) and luminal (grey) breast cancer cell lines with 300 nM BEZ for 8 h or 20 h, respectively. pSTAT5/STAT5 levels were assessed by immunoblotting and quantified by densitometry. pJAK2/JAK2 and IL-8 levels were measured by ELISA. Values from BEZ-treated relative to DMSO cells are given. Data are presented as mean ± SD (n = 3).

1. A combination comprising (a) a phosphoinositide 3-kinase (PI3K) inhibitor compound and (b) a compound which inhibits the interaction between IL-8 and at least one of its receptors, wherein the active ingredients are present in each case in free form or in the form of a pharmaceutically acceptable salt or any hydrate thereof, and optionally at least one pharmaceutically acceptable carrier.

2. A combination according to claim 1 wherein the phosphoinositide 3-kinase inhibitor compound is selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.

3. A combination according to claim 1 wherein the compound inhibits the interaction between IL-8 and at least one of its receptors is selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.

4. (canceled)

5. A combination according to claim 1 wherein the compound which inhibits the interaction of IL-8 with at least one of its receptors is an antibody specifically binding to IL-8.

6. (canceled)

7. A combination according to claim 1 wherein the phosphoinositide 3-kinase inhibitor is COMPOUND C or everolimus.

8-13. (canceled)

14. A combination according to claim 1, wherein said preparation comprises (a) one or more unit dosage forms of phosphoinositide-3 kinase (PI3K) inhibitor and (b) one or more unit dosage forms of a compound which inhibits the interaction between IL-8 and at least one of its receptors.

15. A method of treating a warm-blooded animal having a proliferative disease comprising administering to the animal (a) a phosphoinositide 3-kinase (PI3K) inhibitor compound and (b) a compound which inhibits the interaction between IL-8 and at least one of its receptors, wherein the active ingredients are present in each case in free form or in the form of a pharmaceutically acceptable salt or any hydrate thereof, and optionally at least one pharmaceutically acceptable carrier, in a quantity which is therapeutically effective against said proliferative disease.

16. The method according to claim 15, wherein the phosphoinositide 3-kinase inhibitor compound is selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.

17. The method according to claim 15, wherein the compound inhibits the interaction between IL-8 and at least one of its receptors is selected from the group consisting of an antibody specifically binding to IL-8, an antibody specifically binding to CXCR1, an antibody specifically binding to CXCR2, repertaxin, a siRNA decreasing the expression of IL-8, a siRNA decreasing the expression of CXCR1 and a siRNA decreasing the expression of CXCR2.

18. The method according to claim 15, wherein the compound which inhibits the interaction of IL-8 with at least one of its receptors is an antibody specifically binding to IL-8.

19. The method according to claim 15, wherein the phosphoinositide 3-kinase inhibitor is COMPOUND C.

20. The method according to claim 15, wherein the phosphoinositide 3-kinase inhibitor is everolimus.

21. The method according to any one of claims 15 to 20, wherein the proliferative disease is a solid tumor.

22. The method according to claim 15, wherein the proliferative disease is a breast cancer.

23. The method according to claim 15, wherein the proliferative disease is a metastatic breast cancer or triple-negative breast cancer.


SEQUENCE LISTING (5 sequences)

SEQ ID NO: 1 - RNA, 25 nt, Homo sapiens
  source 1..25: /mol_type=“RNA” /organism=“Homo sapiens”
gcacagaagacggaggaaaugguau

SEQ ID NO: 2 - RNA, 25 nt, Homo sapiens
  source 1..25: /mol_type=“RNA” /organism=“Homo sapiens”
gccuuaaggaauaucuuccaaagaa

SEQ ID NO: 3 - RNA, 21 nt, Homo sapiens
  source 1..21: /mol_type=“RNA” /organism=“Homo sapiens”
aacaagacagcugguaccagg

SEQ ID NO: 4 - RNA, 21 nt, Homo sapiens
  source 1..21: /mol_type=“RNA” /organism=“Homo sapiens”
auucuaucacuagcgugacuu

SEQ ID NO: 5 - DNA, 21 bp, Homo sapiens
  source 1..21: /mol_type=“DNA” /organism=“Homo sapiens”
tggatagttacaactcggctt